(19) United States
(12) Patent Application Publication
(10) Pub. No.: US 2012/0301525 A1
(43) Pub. Date: Nov. 29, 2012

(54) SILICONE OIL EMULSION, METHOD OF PRODUCING SAME, AND SILICONE OIL COMPOSITION

(75) Inventors: Yoshitsugu Morita, Ichihara-shi (JP); Tadashi Takimoto, Ichihara-shi (JP)

(73) Assignee: DOW CORNING TORAY CO., LTD., Chiyoda-ku, Tokyo (JP)

(21) Appl. No.: 13/513,815

(22) PCT Filed: Dec. 2, 2010
(86) PCT No.: PCT/JP2010/072072

§ 371 Date: Aug. 8, 2012

(30) Foreign Application Priority Data
Dec. 4, 2009 (JP) 2009-276771

(51) Int. Cl. A61K 8/06; A61Q 17/04; A61Q 3/02; A61Q 1/00; A61Q 1/04; A61Q 15/00; A61Q 5/00; A61Q 1/10; A61K 8/58; A61Q 19/10; A61Q 1/08; A61Q 5/02; A61Q 5/12; A61Q 5/06; A61Q 13/00; A61Q 5/10; A61K 8/891; A61Q 11/00

(52) U.S. Cl. 424/401; 424/49; 424/59; 424/61; 424/63; 424/64; 424/65; 424/70.1; 424/70.7; 514/63; 512/1; 8/405; 510/159

(57) ABSTRACT

This invention is related to a silicone oil emulsion comprising silicon-containing crosslinked particles in silicone oil droplets that are dispersed in water and have an average particle size of 0.1 to 500 μm wherein the silicon-containing crosslinked particles have an average particle size of 0.05 to 100 μm and are provided by the crosslinking of a crosslinkable composition comprising at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst. The inventive silicone oil emulsion is characterized in that the silicone oil is an alkyl-modified silicone oil that has a silicon-bonded alkyl group having at least 4 carbons. This invention is also related to a silicone oil composition as provided by removing the water from this silicone oil emulsion.

### TECHNICAL FIELD

The present invention relates to a silicone oil emulsion and a method of producing this silicone oil emulsion and to a silicone oil composition.

### BACKGROUND ART

Silicone oil emulsions comprising silicon-containing crosslinked particles in water-dispersed silicone oil droplets are described in Patent Documents 1 and 2. A dimethylpolysiloxane endblocked by the trimethylsiloxy group at both molecular chain terminals is used as the silicone oil here. In addition, Patent Documents 1 and 2 teach the use of these silicone oil emulsions—or the silicone oil compositions obtained by removing the water from these emulsions—as cosmetic materials.

Various properties have come to be required in recent years from the silicone oil emulsions and silicone oil compositions that are used for cosmetic products. For example, the ability to prevent tangled hair and the ability to impart a smooth and silky feel are required in the case of hair cosmetics, while with skin cosmetics the ability to form a suitably spreadable and lustrous film on the skin is required.

[Patent Document 1] JP 2001-139416 A

[Patent Document 2] JP 2001-139819 A

### SUMMARY OF INVENTION

### Technical Problems to be Solved

An object of the present invention is to provide a silicone oil emulsion that contains silicon-containing crosslinked particles in alkyl-modified silicone oil droplets that are dispersed in water. Additional objects of the present invention are to provide a method of producing this silicone oil emulsion and to provide a silicone oil composition in which silicon-containing crosslinked particles are uniformly dispersed in an alkyl-modified silicone oil.

### Solution to Problems

The present inventors have found that a silicone oil emulsion and a silicone oil composition that satisfy various requirements for cosmetic applications can be obtained by using an alkyl-modified silicone oil that has a silicon-bonded alkyl group having at least 4 carbons. The silicone oil emulsion of the present invention comprises silicon-containing crosslinked particles in silicone oil droplets that are dispersed in water and have an average particle size of 0.1 to 500 μm wherein the silicon-containing crosslinked particles have an average particle size of 0.05 to 100 μm and are provided by the crosslinking of a crosslinkable composition comprising at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst, wherein the particle size of the silicon-containing crosslinked particles is less than the particle size of the silicone oil droplets, and is characterized in that the silicone oil is an alkyl-modified silicone oil that has a silicon-bonded alkyl group having at least 4 carbons.

The production method of the present invention is a method of producing a silicone oil emulsion wherein a crosslinkable composition that contains a noncrosslinking silicone oil and that comprises at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst, is caused to undergo a crosslinking reaction in water to produce a silicone oil emulsion that contains silicon-containing crosslinked particles having an average particle size of 0.05 to 100 μm in silicone oil droplets that are dispersed in the water and that have an average particle size of 0.1 to 500 μm wherein the particle size of the silicon-containing crosslinked particles is less than the particle size of the silicone oil droplets, and is characterized in that the silicone oil is an alkyl-modified silicone oil that has a silicon-bonded alkyl group having at least 4 carbons.

The silicone oil composition of the present invention is a silicone oil composition provided by removing the water from a silicone oil emulsion comprising silicon-containing crosslinked particles having an average particle size of 0.05 to 100 in silicone oil droplets that are dispersed in water and have an average particle size of 0.1 to 500 μm wherein the silicon-containing crosslinked particles are provided by the crosslinking of a crosslinkable composition comprising at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst, wherein the particle size of the silicon-containing crosslinked particles is less than the particle size of the silicone oil droplets, and is characterized in that the silicone oil is an alkyl-modified silicone oil that has a silicon-bonded alkyl group having at least 4 carbons.

### Advantageous Effects of Invention

A characteristic feature of the silicone oil emulsion of the present invention is that this silicone oil emulsion contains silicon-containing crosslinked particles in alkyl-modified silicone oil droplets that are dispersed in water and it can thereby improve the properties of cosmetic materials. A characteristic feature of the method of the present invention for producing a silicone oil emulsion is that this method can efficiently produce the indicated silicone oil emulsion. A characteristic feature of the silicone oil composition of the present invention is that it can improve the properties of cosmetic materials because the silicon-containing crosslinked particles in the silicone oil composition are uniformly dispersed in an alkyl-modified silicone oil.

### BEST MODE FOR CARRYING OUT THE INVENTION

The silicone oil emulsion of the present invention is described in detail. The silicone oil emulsion of the present invention contains silicon-containing crosslinked particles in water-dispersed silicone oil droplets and is characterized in that this silicone oil is an alkyl-modified silicone oil that has an at least C₄ alkyl group bonded to silicon.

The silicon-containing crosslinked particles in this emulsion are provided by the crosslinking of a crosslinkable composition that comprises at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst.

The component (A) silicon-free organic compound has at least two aliphatically unsaturated bonds in each molecule. Groups that contain an aliphatically unsaturated bond can be exemplified by groups that reside in molecular chain terminal position and/or molecular chain pendant position, e.g., alkenyl groups such as vinyl, allyl, butenyl, pentenyl, and so forth; alkynyl groups such as ethynyl; and cyclic unsaturated groups such as the norbornene group, the dicyclopentadienyl group, and so forth; and by groups residing within the molecular chain, e.g., enylene groups such as vinylene, propenylene, and so forth. Groups residing in molecular chain terminal position and/or pendant position, e.g., vinyl, allyl, and so forth, are preferred. The state of component (A) at 25° C. is not limited, and, for example, component (A) may be a solid or a liquid at 25° C. wherein the liquid state is preferred. When component (A) is a solid at 25° C., it is preferably dissolved in advance in another component or in an organic solvent. The molecular weight of component (A) is also not limited, but its average molecular weight is preferably 50 to 50,000.

Such component (A) of this invention can be exemplified by dienes, e.g., dienes such as pentadiene, hexadiene, heptadiene, octadiene, nonadiene, cyclopentadiene, cyclooctadiene, and so forth, aromatic dienes such as divinylbenzene and so forth, ethers such as diallyl ether, triethylene glycol divinyl ether, cyclohexanedimethanol divinyl ether, 1,2-divinylglycol, and so forth, esters such as diallyl isophthalate, diallyl phthalate, diallyl terephthalate, diallyl maleate, triallyl trimellitate, and so forth, and by the oligomers provided by polymerizing the preceding. Component (A) can be further exemplified by olefin oligomers that have at least two aliphatically unsaturated bond groups in each molecule and are provided by the polymerization of an olefin such as ethylene, propylene, butene, isobutene, pentene, hexene, and so forth. Component (A) can be further exemplified by the oligomers provided by the polymerization of an alkenyl-functional acrylic monomer such as allyl(meth)acrylate, butenyl(meth)acrylate, methylbutenyl(meth)acrylate, methylpropenyl(meth)acrylate, heptenyl(meth)acrylate, hexenyl(meth)acrylate, and so forth, and by the oligomers provided by the copolymerization of the previously indicated acrylic monomers with, e.g., methyl(meth)acrylate, ethyl(meth)acrylate, butyl(meth)acrylate, ethylhexyl(meth)acrylate, lauryl(meth)acrylate, styrene,α-methylstyrene, maleic acid, vinyl acetate, allyl acetate, and so forth. Component (A) can be further exemplified by the oligomers provided by the reaction of an alkenyl isocyanate, e.g., allyl isocyanate, (meth)acryloyl isocyanate, 2-isocyanatoethyl(meth)acrylate, and so forth, or an alkenyl-functional carboxylic acid anhydride, e.g., itaconic anhydride, maleic anhydride, tetrahydrophthalic anhydride, and so forth, with an oligomer itself provided by the copolymerization of the previously indicated monomers with an hydroxyl-functional acrylic monomer such as 2-hydroxyethyl(meth)acrylate, 2-hydroxypropyl(meth)acrylate, 4-hydroxybutyl(meth)acrylate, and so forth. Component (A) can be further exemplified by the oligomers provided by the reaction of an alkenyl alcohol, e.g., allyl alcohol, butenol, 2-(allyloxy)ethanol, glycerol diallyl ether, cyclohexenemethanol, methylbutenol, oleyl alcohol, and so forth, with an oligomer as provided by the polymerization of an isocyanate group-functional acrylic monomer, e.g., (meth)acryloyl isocyanate, 2-isocyanatoethyl(meth)acrylate, and so forth, or with an oligomer as provided by the copolymerization of such an isocyanate group-functional acrylic monomer with the previously indicated monomers. Component (A) can be further exemplified by the oligomers provided by the reaction of an alkenyl-functional epoxy compound such as glycidyl(meth)acrylate, allyl glycidyl ether, and so forth, with an oligomer as provided by the polymerization of a carboxyl group-functional monomer such as (meth)acrylic acid, itaconic acid, maleic acid, and so forth, or with an oligomer as provided by the copolymerization of such a carboxyl group-functional monomer with the previously indicated monomers. Component (A) can be further exemplified by the polyethers provided by the ring-opening polymerization of allyl glycidyl ether using ethylene glycol as the initiator and by the polyethers provided by the ring-opening polymerization of vinylcyclohexane-1,2-epoxide using, for example, propargyl alcohol, as the initiator. Component (A) can be further exemplified by the alkenyl-functional polyesters provided by the reaction of a polybasic acid, e.g., phthalic anhydride, isophthalic acid, terephthalic acid, adipic acid, azelaic acid, trimellitic acid, and so forth, with an alkenyl alcohol as indicated above and a polyhydric alcohol such as ethylene glycol, propylene glycol, 1,6-hexanediol, diethylene glycol, neopentyl glycol, neopentyl glycol hydroxypivalate, trimethylolpropane, and so forth. Component (A) is preferably a diene or oligomer thereof or a polyether.

The component (B) silicon-containing organic compound has at least two silicon-bonded hydrogen atoms in each molecule. There is no limitation on the viscosity of component (B) at 25° C., but this viscosity is preferably from 1 to 100,000 mPa·s and particularly preferably is from 1 to 10,000 mPa·s. Component (B) can be exemplified by organohydrogenpolysiloxanes and by diorganohydrogensilyl group-containing organic polymers, wherein organohydrogenpolysiloxanes are preferred.

The organohydrogenpolysiloxane of this invention may have, for example, a straight-chain, branched, cyclic, network or mesh, or partially branched straight-chain molecular structure and can be exemplified by the following: a methylhydrogenpolysiloxane endblocked by the trimethylsiloxy group at both molecular chain terminals; a dimethylsiloxane•methylhydrogensiloxane copolymer endblocked by the trimethylsiloxy group at both molecular chain terminals; a dimethylsiloxane•methylhydrogensiloxane•methylphenylsiloxane copolymer endblocked by the trimethylsiloxy group at both molecular chain terminals; a dimethylpolysiloxane endblocked by the dimethylhydrogensiloxy group at both molecular chain terminals; a dimethylsiloxane•methylphenylsiloxane copolymer endblocked by the dimethylhydrogensiloxy group at both molecular chain terminals; a methylphenylpolysiloxane endblocked by the dimethylhydrogensiloxy group at both molecular chain terminals; an organopolysiloxane copolymer comprising the siloxane unit represented by the formula R₃SiO_{1/2}, the siloxane unit represented by the formula R₂HSiO_{1/2}, and the siloxane unit represented by the formula SiO_{4/2}; an organopolysiloxane copolymer comprising the siloxane unit represented by the formula R₂HSiO_{1/2} and the siloxane unit represented by the formula SiO_{4/2}; an organopolysiloxane copolymer comprising the siloxane unit represented by the formula RHSiO_{2/2}, the siloxane unit represented by the formula RSiO_{3/2}, and the siloxane unit represented by the formula HSiO_{3/2}; and mixtures of two or more of the preceding organopolysiloxanes. The group R in the preceding formulas is a non-alkenyl monovalent hydrocarbyl group and can be exemplified by alkyl groups such as methyl, ethyl, propyl, butyl, pentyl, hexyl, heptyl, and so forth; aryl groups such as phenyl, tolyl, xylyl, naphthyl, and so forth; aralkyl groups such as benzyl, phenethyl, and so forth; and halogenated alkyl groups such as chloromethyl, 3-chloropropyl, 3,3,3-trifluoropropyl, and so forth.

The aforementioned diorganohydrogensilyl group-containing organic polymer can be exemplified by the oligomers provided by the copolymerization of a dimethylhydrogensilyl group-containing acrylic monomer, e.g., dimethylhydrogensilyl(meth)acrylate and dimethylhydrogensilylpropyl(meth)acrylate, with a monomer such as methyl(meth)acrylate, ethyl(meth)acrylate, butyl(meth)acrylate, ethylhexyl(meth)acrylate, lauryl(meth)acrylate, styrene, α-methylstyrene, maleic acid, vinyl acetate, allyl acetate, and so forth.

The quantity of component (B) incorporation in the said composition is preferably 0.1 to 500 weight parts, more preferably 0.5 to 500 weight parts, and particularly preferably 1 to 100 weight parts, in each case per 100 weight parts component (A) or per 100 weight parts of the total of components (A) and the herebelow-described component (D). The reasons for this are as follows: crosslinking is inadequate in a composition in which the quantity of component (B) incorporation is below the lower limit on the indicated range, while the excess silicon-bonded hydrogen produces hydrogen gas with a composition in which the upper limit on the indicated range is exceeded.

The component (C) hydrosilylation reaction catalyst is a catalyst that promotes or accelerates the hydrosilylation reaction in the composition under consideration in order to bring about crosslinking. Component (C) can be exemplified by platinum catalysts, rhodium catalysts, and palladium catalysts wherein platinum catalysts are preferred. The platinum catalyst can be exemplified by platinum supported on finely divided silica, platinum supported on finely divided carbon, chloroplatinic acid, alcohol solutions of chloroplatinic acid, platinum/olefin complexes, platinum/alkenylsiloxane complexes, and carbonyl complexes of platinum.

There is no limitation on the quantity of component (C) incorporation in the composition under consideration, and component (C) is incorporated in this composition in an amount sufficient to promote or accelerate the hydrosilylation reaction in the composition. When a platinum catalyst is used for component (C), component (C) is preferably incorporated in an amount that provides from 1×10⁻⁷ to 1×10⁻³ weight part platinum metal in component (C) per 100 weight parts for the total of components (A) and (B) or per 100 weight parts for the total of components (A), (B), and (D). The reasons for this are as follows: crosslinking is not adequately accelerated or promoted in the composition in which the quantity of component (C) incorporation is below the lower limit on the indicated range, while little additional effect accrues when the quantity of incorporation exceeds the upper limit on the indicated range, making this uneconomical.

The component (D) organopolysiloxane, which is an optional component for the composition of this invention, has at least one alkenyl group in each molecule and preferably has at least two alkenyl groups in each molecule. Component (D) functions to improve the compatibility between silicone oils and the obtained silicon-containing crosslinked particles and to impart flexibility and rubbery elasticity. The alkenyl group in component (D) can be exemplified by vinyl, allyl, butenyl, pentenyl, and hexenyl wherein vinyl is preferred. The non-alkenyl silicon-bonded groups in component (D) can be exemplified by monovalent hydrocarbyl groups, e.g., alkyl groups such as methyl, ethyl, propyl, butyl, and so forth; cycloalkyl groups such as cyclopentyl, cyclohexyl, and so forth; aryl groups such as phenyl, tolyl, xylyl, and so forth; aralkyl groups such as benzyl, phenethyl, 3-phenylpropyl, and so forth; and halogenated alkyl groups such as 3-chloropropyl, 3,3,3-trifluoropropyl, and so forth. The molecular structure of component (D) can be exemplified by straight chain, branched, cyclic, network, and partially branched straight chain wherein straight chain is preferred. The viscosity of component (D) at 25° C. is not limited, but is preferably from 20 to 100,000 mPa·s and particularly preferably is from 20 to 10,000 mPa·s.

Component (D) can be exemplified by the following: a dimethylsiloxane•methylvinylsiloxane copolymer endblocked by the trimethylsiloxy group at both molecular chain terminals; a methylvinylpolysiloxane endblocked by the trimethylsiloxy group at both molecular chain terminals; a dimethylsiloxane•methylvinylsiloxane•methylphenylsiloxane copolymer endblocked by the trimethylsiloxy group at both molecular chain terminals; a dimethylpolysiloxane endblocked by the dimethylvinylsiloxy group at both molecular chain terminals; a methylvinylpolysiloxane endblocked by the dimethylvinylsiloxy group at both molecular chain terminals; a dimethylsiloxane•methylvinylsiloxane copolymer endblocked by the dimethylvinylsiloxy group at both molecular chain terminals; a dimethylsiloxane•methylvinylsiloxane•methylphenylsiloxane copolymer endblocked by the dimethylvinylsiloxy group at both molecular chain terminals; an organopolysiloxane copolymer comprising the siloxane unit represented by the formula R₃SiO_{1/2}, the siloxane unit represented by the formula R₂R¹SiO_{1/2}, and the siloxane unit represented by the formula SiO_{4/2}; an organopolysiloxane copolymer comprising the siloxane unit represented by the formula R₂R¹SiO_{1/2} and the siloxane unit represented by the formula SiO_{4/2}; an organopolysiloxane copolymer comprising the siloxane unit represented by the formula RR¹ SiO_{2/2}, the siloxane unit represented by the formula RSiO_{3/2}, and the siloxane unit represented by the formula R¹SiO_{3/2}; and mixtures of two or more of the preceding organopolysiloxanes. The group R in the preceding formulas is a non-alkenyl monovalent hydrocarbyl group and is exemplified by the same groups as indicated above. The group R¹ in the preceding formulas is an alkenyl group and can be exemplified by vinyl, allyl, butenyl, pentenyl, hexenyl, and heptenyl.

Component (D) is incorporated in the composition of this invention in a quantity that provides a value preferably of 0.1:99.9 to 99.9:0.1 for the weight ratio between the contents of components (A) and (D) and particularly preferably a value of 0.5:99.5 to 50:50 for the weight ratio between the contents of components (A) and (D). The reasons for this are as follows: when the component (D) content exceeds the upper limit on the range indicated above, a trend sets in of declining compatibility between the resulting silicon-containing crosslinked particles and silicon-free organic oils; on the other hand, at below the lower limit on the range indicated above, a trend sets in of declining compatibility between the resulting silicon-containing crosslinked particles and silicone oils.

The composition under consideration may incorporate other optional components, for example, a reaction inhibitor in order to modulate the hydrosilylation reaction; a reinforcing filler such as precipitated silica, fumed silica, calcined silica, fumed titanium oxide, and so forth; a nonreinforcing filler such as quartz powder, diatomaceous earth, aluminosilicates, iron oxide, zinc oxide, calcium carbonate, and so forth; and the preceding fillers after surface treatment with an organosilicon compound such as hexamethylsilazane, trimethylchiorosilane, polydimethylsiloxane, polymethylhydrogensiloxane, and so forth.

The average particle size of the silicon-containing crosslinked particle is in the range from 0.05 to 100 μm and is preferably in the range from 0.1 to 100 μm and is particularly preferably in the range from 0.1 to 50 μm. When the average particle size of the silicone oil droplets is in the range from 0.2 to 500 μm, the average particle size of the silicon-containing crosslinked particles is preferably in the range from 0.1 to 100 μm and particularly preferably is in the range from 0.1 to 50 μm. When the average particle size of the silicone oil droplets is in the range from 0.5 to 500 μm, the average particle size of the silicon-containing crosslinked particles is preferably in the range from 0.1 to 100 μm and particularly preferably is in the range from 0.1 to 50 μm. When the average particle size of the silicone oil droplets is in the range from 0.5 to 200 μm, the average particle size of the silicon-containing crosslinked particles is preferably in the range from 0.1 to 100 μm and particularly preferably is in the range from 0.1 to 50 μm. The reasons for this are as follows: it is quite difficult to produce silicon-containing crosslinked particles that have an average particle size below the lower limit on the indicated range, while the emulsion has a diminished stability when the silicon-containing crosslinked particles exceed the upper limit on the indicated range. The particle size of the silicon-containing crosslinked particles in the emulsion must of course be smaller than the particle size of the silicone oil droplets. The shape of the silicon-containing crosslinked particles can be exemplified by spherical, spindle shaped, flat, or irregular and is preferably spherical. The properties of the silicon-containing crosslinked particle are preferably elastomeric, e.g., gel-like, rubbery, and so forth.

The alkyl group in the alkyl-modified silicone oil in the emulsion under consideration contains at least 4 carbons and preferably contains at least 6 carbons and particularly preferably contains at least 8 carbons. While there is no limitation on the upper limit on the number of carbons in this alkyl group, this alkyl group preferably contains no more than 30 carbons and particularly preferably contains no more than 20 carbons. This alkyl group can be exemplified by butyl, pentyl, hexyl, heptyl, octyl, nonyl, and decyl. The molecular structure of this alkyl-modified silicone oil is not limited and can be exemplified by straight chain, branched chain, and cyclic. The silicon-bonded groups in this alkyl-modified silicone oil other than the at least C₄ alkyl can be exemplified by alkyl groups having not more than 3 carbons, e.g., methyl, ethyl, and propyl; alkenyl groups such as vinyl, allyl, and butenyl; aryl groups such as phenyl, tolyl, and xylyl; halogenated alkyl groups such as 3,3,3-trifluoropropyl; alkoxy groups such as methoxy, ethoxy, and propoxy; and also by the hydrogen atom and the hydroxyl group. Methyl and phenyl are preferred.

There are no particular limitations on the viscosity of this alkyl-modified silicone oil at 25° C., but the viscosity at 25° C. is preferably in the range from 1 to 100,000,000 mPa·s and particularly preferably is in the range from 2 to 10,000,000 mPa·s. This alkyl-modified silicone oil is preferably compatible with the previously described crosslinkable composition and also preferably does not participate in the crosslinking reaction in this crosslinkable composition. In specific terms, preferably neither alkenyl nor silicon-bonded hydrogen is present in the molecule.

This alkyl-modified silicone oil can be represented, for example, by the following average formula.

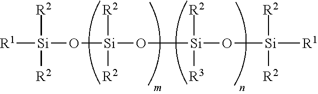

R¹ this formula is an alkyl group having no more than 3 carbons, an alkyl group having at least 4 carbons, an aryl group, or a halogenated alkyl group and can be exemplified by the same groups as provided above. R² in the formula is an alkyl group having no more than 3 carbons, an aryl group, or a halogenated alkyl group and can be exemplified by the same groups as provided above. R³ in the formula is an alkyl group having at least 4 carbons and can be exemplified by the same groups as provided above. m and n in the formula are each zero or a positive number, wherein when n is zero at least one of R¹ should be an alkyl group having at least 4 carbons.

The alkyl-modified silicone oil in the emulsion is dispersed in droplet form in the water. The average particle size of these droplets is in the range from 0.1 to 500 μm and is preferably in the range from 0.2 to 500 μm, more preferably in the range from 0.5 to 500 μm, and particularly preferably in the range from 0.5 to 200 μm. The reasons for this are as follows: it is quite difficult to prepare an emulsion in which the droplets have an average particle size below the lower limit on the indicated range, while an emulsion in which the upper limit on the indicated range is exceeded has a reduced stability.

The method of the present invention for producing the silicone oil emulsion is described in detailed herebelow.

The crosslinkable composition used by the production method of the present invention comprises at least the previously described components (A) to (C), as necessary component (D), and optional components as previously described. In the execution of this production method, the crosslinkable composition in which component (C) has already been incorporated may be dispersed in the water, or the crosslinkable composition exclusive of component (C) may be dispersed in the water and component (C) may then be subsequently added to the water to prepare the hydrosilylation reaction catalyst-containing crosslinkable composition in the water. The use is preferred in the latter case of a water-based dispersion in which component (C) is dispersed into an average particle size of not more than 1 μm.

The alkyl-modified silicone oil must be incorporated in the composition under consideration in a quantity that exceeds the quantity of the alkyl-modified silicone oil that can be retained in the crosslinked product from the crosslinkable composition, i.e., the alkyl-modified silicone oil must be incorporated in a quantity that exceeds the quantity of the alkyl-modified silicone oil that this crosslinked product can contain. The amount that can be retained will vary as a function of the particular crosslinkable composition+alkyl-modified silicone oil combination, but as a general matter the alkyl-modified silicone oil is incorporated preferably in the range from 200 to 5,000 weight parts and particularly preferably in the range from 250 to 2,000 weight parts, in each case per 100 weight parts of the crosslinkable composition.

The production method under consideration is characterized by dispersing the alkyl-modified silicone oil-containing crosslinkable composition in water and thereafter bringing about the crosslinking reaction in the crosslinkable composition. The method of dispersing this crosslinkable composition in water can be exemplified by dispersing this composition in the water using a device such as a homomixer, paddle mixer, Henschel mixer, Homo Disper, colloid mill, propeller stirrer, homogenizer, inline continuous emulsifier, ultrasound emulsifier, vacuum kneader/mixer, and so forth.

There is no limitation on the quantity of water used in the production method under consideration, but the water is preferably used within the range from 5 to 99 weight % of the emulsion as a whole and is particularly preferably used within the range from 10 to 80 weight % of the emulsion as a whole.

A nonionic surfactant, e.g., a polyoxyalkylene alkyl ether, polyoxyalkylene alkylphenol, polyoxyalkylene alkyl ester, polyoxyalkylene sorbitan ester, polyethylene glycol, polypropylene glycol, diethylene glycol, ethylene oxide adduct on trimethylnonanol, and so forth; an anionic surfactant, e.g., hexylbenzenesulfonic acid, octylbenzenesulfonic acid, decylbenzenesulfonic acid, dodecylbenzenesulfonic acid, cetylbenzenesulfonic acid, myristylbenzenesulfonic acid, and their sodium salts and so forth; or a cationic surfactant, e.g., octyltrimethylammonium hydroxide, dodecyltrimethylammonium hydroxide, hexadecyltrimethylammonium hydroxide, octyldimethylbenzylammonium hydroxide, decyldimethylbenzylammonium hydroxide, dioctadecyldimethylammonium hydroxide, beef tallow trimethylammonium hydroxide, cocotrimethylammonium hydroxide, and so forth, is preferably used in order to bring about a highly stable dispersion of the crosslinkable composition in the water, and the use of a nonionic surfactant is particularly preferred. This surfactant is used preferably at from 0.1 to 20 weight parts and particularly preferably at from 0.5 to 10 weight parts, in each case per 100 weight parts of the crosslinkable composition containing the noncrosslinking alkyl-modified silicone oil. A thickener such as 2-phenoxyethanol, carboxymethyl cellulose, xanthan gum, and so forth, may also be incorporated in order to improve the stability of the emulsion.

The average particle size of the water-dispersed crosslinkable composition in the production method under consideration must be in the range from 0.1 to 500 μm and is preferably in the range from 0.2 to 500 μm, more preferably in the range from 0.5 to 500 μm, and particularly preferably in the range from 0.5 to 200 μm. The reasons for this are as follows: it is quite difficult to produce an emulsion in which the average particle size of the water-dispersed crosslinkable composition is below the lower limit on the indicated range, while an emulsion that exceeds the upper limit on the indicated range has a reduced stability.

The crosslinking reaction in the water-dispersed crosslinkable composition can be brought about by heating the thusly produced emulsion of the crosslinkable composition or by allowing this emulsion to stand at room temperature.

The silicone oil composition of the present invention is described in detail in the following.

The silicone oil composition of the present invention is characteristically provided by removing the water from the silicone oil emulsion containing silicon-containing crosslinked particles in water-dispersed alkyl-modified silicone oil droplets wherein the silicon-containing crosslinked particles have been provided by the crosslinking of the crosslinkable composition comprising at least the previously described components (A) , to (C), as necessary component (D), and the previously described optional components. This silicone oil emulsion is a silicone oil emulsion produced as described in the preceding.

There are no limitations on the method of producing the silicone oil composition, and it can be produced by removing the water using a means such as subjecting the previously described silicone oil emulsion to air drying, drying in a hot air current, vacuum drying, drying with the application of heat, and so forth. The silicon-containing crosslinked particles are uniformly dispersed in the alkyl-modified silicone oil in this silicone oil composition, and the state of this silicone oil composition can be exemplified by liquid, cream, paste, and grease.

The silicone oil emulsion of the present invention and the silicone oil composition of the present invention are well suited for application as a cosmetic material or as an ingredient for a cosmetic material. The type of cosmetic material here can be exemplified by cleansing cosmetics such as soaps, body shampoos, facial cleansing creams, and so forth; basic cosmetics such as face lotions, creams•milky lotions, packs, and so forth; base make-up cosmetics such as facial powders, foundations, and so forth; facial cosmetics such as lipsticks, blushes, eye shadows, eye liners, mascaras, and so forth; make-up cosmetics such as nail polishes and so forth; hair cosmetics such as shampoos, hair rinses, hair conditioners, hair treatments, set lotions, blow styling lotions, hair sprays, foam styling agents, gel styling agents, hair liquids, hair tonics, hair creams, hair growth agents, hair restoration agents, hair dyes, hair styling agents, and so forth; aromatic cosmetics such as perfumes, eau de colognes, and so forth; toothpastes; bath agents; and specialty cosmetics such as depilatories, shaving lotions, antiperspirants•deodorants, and sunscreens. Skin cosmetics such as basic cosmetics and make-up cosmetics as well as hair cosmetics are preferred examples. These cosmetics can be formulated as, for example, water-based liquids, oil-based liquids, emulsions, creams, foams, semi-solids, solids, and powders. These cosmetic products may also be used as sprays.

The following, for example, may be incorporated as cosmetic ingredients when a cosmetic material is produced using this silicone oil emulsion or silicone oil composition: waxes such as carnauba wax, candelilla wax, Rhus succedanea fruit wax, spermaceti, jojoba wax, montan wax, beeswax, and so forth; fats and oils such as liquid paraffin, isoparaffin, hexyl laurate, isopropyl myristate, myristyl myristate, cetyl myristate, 2-octyldodecyl myristate, isopropyl palmitate, 2-ethylhexyl palmitate, butyl stearate, decyl oleate, 2-octyldodecyl oleate, myristyl lactate, cetyl lactate, lanolin acetate, stearyl alcohol, cetostearyl alcohol, oleyl alcohol, avocado oil, almond oil, olive oil, cacao oil, jojoba oil, sesame oil, safflower oil, soy oil, camellia oil, squalane, persic oil, castor oil, mink oil, cottonseed oil, coconut oil, egg yolk oil, lard, and so forth; glycol ester oils such as polypropylene glycol monooleate, neopentyl glycol 2-ethylhexanoate, and so forth; polyhydric alcohol ester oils such as triisostearin, cocofatty acid triglycerides, and so forth; polyoxyalkylene ether oils such as polyoxyethylene lauryl ether, polyoxypropylene cetyl ether; and so forth; and silicone oils such as dimethylpolysiloxane, methylphenylpolysiloxane, octamethyltetracyclosiloxane, decamethylcyclopentasiloxane, polyether-modified silicone oil, amino-modified silicone oil, and so forth.

Other optional cosmetic ingredients can be exemplified by humectants such as glycerol, propylene glycol, 1,3-butylene glycol, polyethylene glycol, sodium d,l-pyrrolidonecarboxylate, sodium lactate, sorbitol, sodium hyaluronate, and so forth; surfactants such as anionic surfactants, e.g., higher fatty acid soaps, higher alcohol sulfate ester salts, N-acylglutamate salts, phosphate ester salts, and so forth, cationic surfactants, amphoteric surfactants, e.g., betaine types, amino acid types, imidazoline types, lecithin types, and so forth, and nonionic surfactants such as polyhydric alcohol esters, ethylene oxide condensates, and so forth; pigments such as colored pigments, e.g., iron oxide and so forth, white pigments, e.g., zinc oxide, titanium oxide, zirconium oxide, and so forth, and extender pigments such as mica, talc, sericite, and so forth; thickeners such as carrageenan, alginic acid, gum arabic, tragacanth, pectin, starch, xanthan gum, guar gum, carboxymethyl cellulose, carboxyvinyl polymers, polyvinyl alcohol, polyvinylpyrrolidone, hydroxyethyl cellulose, polyoxyethylene glycol distearate, sodium polyalginate, polyethylene glycol, and so forth; ultraviolet absorbers such as benzophenone derivatives such as 2-hydroxy-4-methoxybenzophenone and so forth, benzotriazole derivatives such as 2-(2′-hydroxy-5′-methylphenyl)benzotriazole and so forth, and cinnamate esters; anti-inflammatories such as potassium glycyrrhizate, tocopherol acetate, and so forth; preservatives such as methylparaben, butylparaben, and so forth; antimicrobials such as triclosan, trichlorocarban, and so forth; and antioxidants such as BHA, BHT, γ-oryzanol, and so forth.

In particular, the following, for example, can also be incorporated as cosmetic ingredients when the cosmetic takes the form of a hair cosmetic: surfactants such as nonionic surfactants, e.g., glycerol fatty acid esters such as glycerol monostearate, sorbitan fatty acid esters such as sorbitan monopalmitate, polyoxyethylene alkyl ethers such as polyoxyethylene cetyl ether, polyoxyethylene fatty acid esters such as polyoxyethylene stearate and polyoxyethylene sorbitan monolaurate, as well as polyoxyethylene alkylphenyl ethers, polyoxyethylene castor oil, polyoxyethylene hardened castor oil, and the alkylolamides of fatty acids, cationic surfactants such as monoalkyltrimethylammonium salts, e.g., stearyltrimethylammonium chloride and behenyltrimethylammonium chloride, and dialkyldimethylammonium salts, e.g., distearyldimethylammonium chloride and dibehenyldimethylammonium chloride, and amphoteric surfactants; film-forming agents such as the polymers of (meth)acrylic-type radically polymerizable monomers and their copolymers with silicone compounds, poly(N-acylalkyleneimine), poly(N-methylpyrrolidone), silicone resins modified by a fluorine-containing organic group and/or the amino group, and unmodified silicone resins; anti-dandruff agents such as sulfur, selenium sulfide, zinc pyrithione, octopirox, zinc pyridium-1-thiol-N-oxide, salicylic acid, 2,4,4′-trichloro-2′-hydroxydiphenyl ether, 1-hydroxy-2-pyridone compounds, and so forth; tactile feel improvers such as squalane, lanolin, perfluoropolyether, cationic polymers, and so forth; antifreezes such as ethanol, isopropyl alcohol, 1,3-butylene glycol, ethylene glycol, propylene glycol, glycerol, and so forth; chelating agents such as ethylenediaminetetraacetic acid, citric acid, ethane-1-hydroxy-1,1-diphosphonic acid, and salts of the preceding; colorants such as pearlescent agents, pigments, dyes, and so forth; vitamins; hair restoration agents; hormones; fragrances; pH modifiers; propellants; and the components described in the Encyclopedia of Shampoo Ingredients (Micelle Press, 1985).

There is no limitation on the quantity of incorporation of the silicone oil emulsion or silicone oil composition when this emulsion or composition is used to produce a cosmetic material, but the emulsion or composition is preferably used at 0.1 to 99.9 weight % in the cosmetic material and particularly preferably at 0.5 to 99 weight % in the cosmetic material, in each case calculated on the basis of the components other than water, i.e., calculated on the basis of the solids fraction. The reasons for this are as follows: when the quantity of incorporation of the silicone oil emulsion or silicone oil composition exceeds the upper limit on the previously indicated range, the effect as a cosmetic material tends to be lost; on the other hand, obtaining improvements in, for example, the use sensation, tends to be problematic at below the lower limit on the previously indicated range.

### EXAMPLES

The silicone oil emulsion of the present invention, the method according to the present invention of producing this silicone oil emulsion, and the silicone oil composition of the present invention are described in detail using examples. The viscosity values given in the examples were measured at 25° C. The average particle size and stability of the silicone oil emulsions, the average particle size and dispersibility of the crosslinked silicone particles, and the viscoelasticity of the silicone oil compositions were determined as follows.

[Average Particle Size of the Silicone Oil Emulsion]

The silicone oil emulsion was measured using an LA-750 laser diffraction particle size distribution analyzer from Horiba, Ltd., and the obtained median diameter, which was the particle diameter corresponding to 50% in the cumulative distribution, was used as the average particle size.

[Stability of the Silicone Oil Emulsion]

180 mL of the silicone oil emulsion was sealed in a 225-mL glass bottle having a depth of 105 mm and a mouth diameter of 50 mm and was then held at quiescence for 1 week at room temperature. After standing, the thickness of the aqueous layer that had separated from the emulsion was measured.

[Average Particle Size of the Crosslinked Silicone Particles]

The silicone oil emulsion was air dried on a glass plate, and a sample was prepared by collecting the crosslinked silicone particles under a stereomicroscope. This sample was observed with an electron microscope, and the average particle size was determined from 10 particle diameters.

[Dispersibility of the Crosslinked Silicone Particles]

The silicone oil emulsion was air dried on a glass plate and the shape of the crosslinked silicone particles, their aggregation status, and their distribution were observed with a stereomicroscope. A score of “+” was rendered when all of the crosslinked silicone particles were dispersed as primary particles; a score of “×” was rendered when aggregated particles of several hundred micrometers were present or when primary particles of 500 μm or more were present; while a state intermediate between the preceding was scored with a “Δ”.

[Viscoelasticity of the Silicone Oil Composition]

The storage modulus G′ (Pa), the loss modulus G″ (Pa), and the loss tangent tan δ were measured on the silicone oil composition using an ARES viscoelasticity analyzer from Rheometric Scientific. The measurement conditions were as follows: room temperature, 25 mm parallel plates, gap=0.5 to 0.6 mm, strain=10%, oscillation rate=0.01 to 50 Hz.

### Practical Example 1

The following were mixed to produce a crosslinkable composition: 33.00 weight parts of a dimethylsiloxane•methylvinylsiloxane copolymer endblocked by the dimethylvinylsiloxy group at both molecular chain terminals and having a viscosity of 400 mPa·s and a vinyl content of 1.18 weight %, 6.38 weight parts of a dimethylsiloxane•methylhydrogensiloxane copolymer endblocked by the trimethylsiloxy group at both molecular chain terminals and having a viscosity of 50 mPa·s and a silicon-bonded hydrogen content of 0.43 weight %, 0.62 weight part 1,5-hexadiene, and 60 weight parts of an alkyl-modified silicone oil having the following average formula

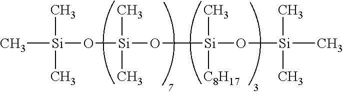

and a viscosity of 30 mPa·s.

An aqueous solution was prepared by dissolving 1.6 weight parts of a olyoxyethylene alkyl ether having an HLB of 14.5 and 1.6 weight parts 2-phenoxyethanol in 96.8 weight parts pure water, and 29.5 weight parts of this previously prepared aqueous solution was added to the composition prepared as above. After emulsification using a colloid mill, an additional 27.6 weight parts pure water was added to give a water-based emulsion of the crosslinkable composition.

To this emulsion was added a water-based emulsion of a platinum catalyst in which the main component was a 1,3-divinyl-1,1,3,3-tetramethyldisiloxane complex of platinum and mixing to uniformity was performed. This water-based platinum catalyst emulsion had an average platinum catalyst particle size of 0.05 μm and a platinum metal concentration of 0.05 weight %, and it was added in an amount that provided 10 weight-ppm platinum metal with reference to the crosslinkable composition in the water-based emulsion of the crosslinkable composition.

The crosslinkable composition was crosslinked via the hydrosilylation reaction by holding the water-based emulsion of the crosslinkable composition for 1 day at 50° C.; this produced an alkyl-modified silicone oil emulsion that contained rubbery silicon-containing crosslinked particles in alkyl-modified silicone oil droplets that were dispersed in the water. The properties of this emulsion are given in Table 1. A portion of this emulsion was collected and hair was immersed therein followed by drying. The hair resisted tangling, and a silky smooth feel was imparted to the hair.

This emulsion was then introduced into a Model HV-030 Vacuum Mixer combination mixer from the STM Co., Ltd. While stirring at an anchor mixer rotation rate of 90 rpm and a disperser rotation rate of 1,000 rpm, the water was removed by reducing the pressure while raising the temperature to 75 to 85° C. over 1 to 2 hours and holding for 1 hour at 50 mmHg or below. This was followed by cooling to room temperature to obtain a liquid alkyl-modified silicone oil composition. When this composition was observed with a stereomicroscope, the silicone rubber particles were found to be uniformly dispersed in the alkyl-modified silicone oil and to have a spherical shape. The properties of this composition are given in Table 1. A portion of this composition was collected and spread on the back of the hand with a finger: it spread very smoothly and demonstrated a suitable spreadability. In addition, a lustrous and water-repellent film could be formed on the back of the hand.

### Practical Example 2

The following were mixed to produce a crosslinkable composition: 10.81 weight parts of a dimethylsiloxane•methylvinylsiloxane copolymer endblocked by the dimethylvinylsiloxy group at both molecular chain terminals and having a viscosity of 400 mPa·s and a vinyl content of 1.18 weight %, 2.10 weight parts of a dimethylsiloxane•methylhydrogensiloxane copolymer endblocked by the trimethylsiloxy group at both molecular chain terminals and having a viscosity of 50 mPa·s and a silicon-bonded hydrogen content of 0.43 weight %, 7.10 weight parts of a polypropylene oxide endblocked by the allyl group at both molecular chain terminals and having a viscosity of 400 mPa·s and a number-average molecular weight of 3,000, and 80 weight parts of an alkyl-modified silicone oil having the following average formula

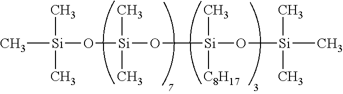

and a viscosity of 30 mPa·s.

An aqueous solution was prepared by dissolving 1.6 weight parts of a polyoxyethylene alkyl ether having an HLB of 14.5 and 1.6 weight parts 2-phenoxyethanol in 96.8 weight parts pure water, and 29.5 weight parts of this previously prepared aqueous solution was added to the composition prepared as above. After emulsification using a colloid mill, an additional 27.6 weight parts pure water was added to give a water-based emulsion of the crosslinkable composition.

To this emulsion was added a water-based emulsion of a platinum catalyst in which the main component was a 1,3-divinyl-1,1,3,3-tetramethyldisiloxane complex of platinum and mixing to uniformity was performed. This water-based platinum catalyst emulsion had an average platinum catalyst particle size of 0.05 μm and a platinum metal concentration of 0.05 weight %, and it was added in an amount that provided 40 weight-ppm platinum metal with reference to the crosslinkable composition in the water-based emulsion of the crosslinkable composition.

The crosslinkable composition was crosslinked via the hydrosilylation reaction by holding the water-based emulsion of the crosslinkable composition for 1 day at 50° C.; this produced an alkyl-modified silicone oil emulsion that contained rubbery silicon-containing crosslinked particles in alkyl-modified silicone oil droplets that were dispersed in the water. The properties of this emulsion are given in Table 1. A portion of this emulsion was collected and hair was immersed therein followed by drying. The hair resisted tangling, and a silky smooth feel was imparted to the hair.

This emulsion was then introduced into a Model HV-030 Vacuum Mixer combination mixer from the STM Co., Ltd. While stirring at an anchor mixer rotation rate of 90 rpm and a disperser rotation rate of 1,000 rpm, the water was removed by reducing the pressure while raising the temperature to 75 to 85° C. over 1 to 2 hours and holding for 1 hour at 50 mmHg or below. This was followed by cooling to room temperature to obtain a liquid alkyl-modified silicone oil composition. When this composition was observed with a stereomicroscope, the rubbery silicon-containing crosslinked particles were found to be uniformly dispersed in the alkyl-modified silicone oil and to have a spherical shape. The properties of this composition are given in Table 1. A portion of this composition was collected and spread on the back of the hand with a finger: it spread very smoothly and demonstrated a suitable spreadability. In addition, a lustrous and water-repellent film could be formed on the back of the hand.

### Comparative Example 1

A silicone oil emulsion containing silicon-containing crosslinked particles and a silicone oil composition crumb containing silicon-containing crosslinked particles were produced proceeding as in Example 1, but in this case replacing the alkyl-modified silicone oil used in Example 1 with the same amount of a dimethylpolysiloxane endblocked by the trimethylsiloxy group at both molecular chain terminals and having a viscosity of 6 mPa·s.

### Comparative Example 2

A silicone oil emulsion containing silicon-containing crosslinked particles was produced proceeding as in Example 2, but in this case replacing the alkyl-modified silicone oil used in Example 2 with the same amount of a dimethylpolysiloxane endblocked by the trimethylsiloxy group at both molecular chain terminals and having a viscosity of 6 mPa·s. When the attempt was made to remove the water from this emulsion as in Example 2, the silicon-containing crosslinked particles separated from the silicone oil and a uniform composition could not be obtained.

### Comparative Example 3

The following were mixed to produce a crosslinkable composition: 82.51 weight parts of a dimethylsiloxane•methylvinylsiloxane copolymer endblocked by the dimethylvinylsiloxy group at both molecular chain terminals and having a viscosity of 400 mPa·s and a vinyl content of 1.18 weight %, 15.94 weight parts of a dimethylsiloxane•methylhydrogensiloxane copolymer endblocked by the trimethylsiloxy group at both molecular chain terminals and having a viscosity of 50 mPa·s and a silicon-bonded hydrogen content of 0.43 weight %, and 1.56 weight parts 1,5-hexadiene.

An aqueous solution was prepared by dissolving 1.6 weight parts of a polyoxyethylene alkyl ether having an HLB of 14.5 and 1.6 weight parts 2-phenoxyethanol in 96.8 weight parts pure water, and 29.5 weight parts of this previously prepared aqueous solution was added to the composition prepared as above. After emulsification using a colloid mill, an additional 27.6 weight parts pure water was added to give a water-based emulsion of the crosslinkable composition.

To this emulsion was added a water-based emulsion of a platinum catalyst in which the main component was a 1,3-divinyl-1,1,3,3-tetramethyldisiloxane complex of platinum and mixing to uniformity was performed. This water-based platinum catalyst emulsion had an average platinum catalyst particle size of 0.05 μm and a platinum metal concentration of 0.05 weight %, and it was added in an amount that provided 10 weight-ppm platinum metal with reference to the crosslinkable composition in the water-based emulsion of the crosslinkable composition.

The crosslinkable composition was crosslinked via the hydrosilylation reaction by holding the water-based emulsion of the crosslinkable composition for 1 day at room temperature; this produced a water-based suspension of silicon-containing crosslinked particles dispersed in the water. This suspension was air dried for 1 week at room temperature to produce silicon-containing crosslinked particles.

20 weight parts of these silicon-containing crosslinked particles and 80 weight parts of an alkyl-modified silicone oil having the following average formula

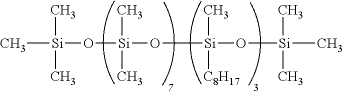

and a viscosity of 30 mPa·s were mixed for 10 minutes at 300 rpm using a blade-type stirrer (Three-One motor machine). The mixture contained aggregated particles that could be felt with the fingers and a uniform composition was thus not obtained. When observation was performed after 1 week, the silicon-containing crosslinked particles were found to have undergone sedimentation.

### Comparative Example 4

The following were mixed to produce a crosslinkable composition: 54.10 weight parts of a dimethylsiloxane•methylvinylsiloxane copolymer endblocked by the dimethylvinylsiloxy group at both molecular chain terminals and having a viscosity of 400 mPa·s and a vinyl content of 1.18 weight %, 10.45 weight parts of a dimethylsiloxane•methylhydrogensiloxane copolymer endblocked by the trimethylsiloxy group at both molecular chain terminals and having a viscosity of 50 mPa·s and a silicon-bonded hydrogen content of 0.43 weight %, and 35.45 weight parts of a polypropylene oxide capped by the allyl group at both molecular chain terminals and having a viscosity of 400 mPa·s and a number-average molecular weight of 3,000.

An aqueous solution was prepared by dissolving 1.6 weight parts of a polyoxyethylene alkyl ether having an HLB of 14.5 and 1.6 weight parts 2-phenoxyethanol in 96.8 weight parts pure water, and 46.5 weight parts of this previously prepared aqueous solution was added to the composition prepared as above. After emulsification using a colloid mill, an additional 96.0 weight parts pure water was added to give a water-based emulsion of the crosslinkable composition.

To this emulsion was added a water-based emulsion of a platinum catalyst in which the main component was a 1,3-divinyl-1,1,3,3-tetramethyldisiloxane complex of platinum and mixing to uniformity was performed. This water-based platinum catalyst emulsion had an average platinum catalyst particle size of 0.05 μm and a platinum metal concentration of 0.05 weight %, and it was added in an amount that provided 40 weight-ppm platinum metal with reference to the crosslinkable composition in the water-based emulsion of the crosslinkable composition.

The crosslinkable composition was crosslinked via the hydrosilylation reaction by holding the water-based emulsion of the crosslinkable composition for 1 day at room temperature; this produced a water-based suspension of silicon-containing crosslinked particles dispersed in the water. This suspension was air dried for 1 week at room temperature to produce silicon-containing crosslinked particles.

20 weight parts of these silicone rubber particles and 80 weight parts of an alkyl-modified silicone oil having the following average formula

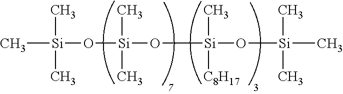

and a viscosity of 30 mPa·s were mixed for 10 minutes at 300 rpm using a blade-type stirrer (Three-One motor machine). The mixture contained aggregated particles that could be felt with the fingers and a uniform composition was thus not obtained. When observation was performed after 1 week, the silicone rubber particles were found to have undergone sedimentation.

TABLE 1

| | | classification | | |
|---|---|---|---|---|
| | | Practical | Practical | Comparative |
| item | | Example 1 | Example 2 | Example 1 |
| silicone oil emulsion | | | | |
| average particle size | | 2.8 | 4.0 | 3.0 |
| (μm) | | | | |
| stability | | + | + | + |
| silicon-containing | | | | |
| crosslinked particles | | | | |
| average particle size | | 2.2 | 3.1 | 2.3 |
| (μm) | | | | |
| dispersibility | | + | + | + |
| viscoelasticity of the silicone | | | | |
| oil composition | | | | |
| G′ | 0.1 Hz | 71 | 1203 | 7665 |
| | 1.0 Hz | 88 | 1347 | 17023 |
| G″ | 0.1 Hz | 44 | 130 | 12873 |
| | 1.0 Hz | 63 | 260 | 20086 |
| tan δ | 0.1 Hz | 0.62 | 0.11 | 1.7 |
| | 1.0 Hz | 0.72 | 0.19 | 1.2 |

### INDUSTRIAL APPLICABILITY

The silicone oil emulsion of the present invention is well qualified for use as a cosmetic ingredient or a cosmetic material. This emulsion is particularly well qualified for application as a hair cosmetic because it makes hair resistant to tangling and can impart a silky smooth feel to hair. In addition, the removal of the water from this emulsion can produce a silicone oil composition in which silicon-containing crosslinked particles are uniformly dispersed in an alkyl-modified silicone oil. This composition is well qualified for use as a lubricating agent, as an additive for resins and plastics, and as a cosmetic ingredient and cosmetic material. This composition is particularly well qualified for application to skin cosmetics because it can provide a suitable spreadability during application to the skin and post-application can form a lustrous and water-repellent film.

The silicone oil emulsion of the present invention and silicone oil composition of the present invention, when used as a cosmetic ingredient or cosmetic material, exhibit an excellent compatibility with high polarity cosmetic ingredients and in particular exhibit an excellent lubricity when in contact with water when the silicon-containing crosslinked particle contains a polyether chain.

1. A silicone oil emulsion comprising silicon-containing crosslinked particles in silicone oil droplets that are dispersed in water and have an average particle size of 0.1 to 500 μm wherein the silicon-containing crosslinked particles have an average particle size of 0.05 to 100 μm and are provided by the crosslinking of a crosslinkable composition comprising at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst, wherein the particle size of the silicon-containing crosslinked particles is less than the particle size of the silicone oil droplets, the silicone oil emulsion being characterized in that the silicone oil is an alkyl-modified silicone oil that has a silicon-bonded alkyl group having at least 4 carbons.

2. The silicone oil emulsion according to claim 1, wherein the crosslinkable composition further comprises (D) an organopolysiloxane that has at least one alkenyl group in each molecule.

3. The silicone oil emulsion according to claim 1, wherein component (A) is a diene or oligomer thereof or is a polyether.

4. The silicone oil emulsion according to claim 1, wherein component (B) is an organohydrogenpolysiloxane.

5. The silicone oil emulsion according to claim 1, wherein the alkyl-modified silicone oil is represented by the following average formula 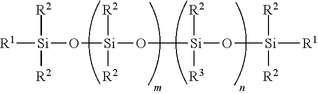
wherein R¹ is an alkyl group having no more than 3 carbons, an alkyl group having at least 4 carbons, an aryl group, or a halogenated alkyl group; R² is an alkyl group having no more than 3 carbons, an aryl group, or a halogenated alkyl group; R³ is an alkyl group having at least 4 carbons; and m and n are each zero or a positive number wherein when n is zero, at least one of R¹ is an alkyl group having at least 4 carbons.

6. A cosmetic ingredient or a cosmetic material comprising the silicone oil emulsion according to claim 1.

7. A production method of a silicone oil emulsion wherein the emulsion is provided by a crosslinking reaction of a crosslinkable silicone composition containing a non-crosslinking silicone oil in water and that comprises at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst and the emulsion contains crosslinked silicone particles having an average particle size of 0.05 to 100 μm in silicone oil droplets that are dispersed in the water as particles having an average particle size of 0.1 to 500 μm wherein the particle size of the crosslinked silicone particles is less than the particle size of the silicone oil droplets, the production method of the silicone oil emulsion is characterized in that the silicone oil is an alkyl-modified silicone oil that has a silicon-bonded alkyl group having at least 4 carbons.

8. The production method according to claim 7, wherein the crosslinkable composition further comprises (D) an organopolysiloxane that has at least one alkenyl group in each molecule.

9. The production method according to claim 7, wherein component (A) is a diene or oligomer thereof or is a polyether.

10. The production method according to claim 7, wherein component (B) is an organohydrogenpolysiloxane.

11. The production method according to claim 7, wherein the alkyl-modified silicone oil is represented by the following average formula 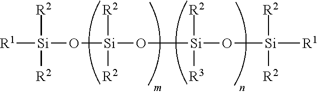
wherein R¹ is an alkyl group having no more than 3 carbons, an alkyl group having at least 4 carbons, an aryl group, or a halogenated alkyl group; R² is an alkyl group having no more than 3 carbons, an aryl group, or a halogenated alkyl group; R³ is an alkyl group having at least 4 carbons; and m and n are each zero or a positive number wherein when n is zero, at least one of R¹ is an alkyl group having at least 4 carbons.

12. A silicone oil composition provided by removing the water from a silicone oil emulsion comprising silicon-containing crosslinked particles having an average particle size of 0.05 to 100 μm in silicone oil droplets that are dispersed in water and have an average particle size of 0.1 to 500 μm wherein the silicon-containing crosslinked particles are provided by the crosslinking of a crosslinkable composition comprising at least (A) a silicon-free organic compound that has at least two aliphatically unsaturated bonds in each molecule, (B) a silicon-containing organic compound that has at least two silicon-bonded hydrogen atoms in each molecule, and (C) a hydrosilylation reaction catalyst, wherein the particle size of the silicon-containing crosslinked particles is less than the particle size of the silicone oil droplets, the silicone oil composition being characterized in that the silicone oil is an alkyl-modified silicone oil that has an at least C₄ alkyl group bonded to silicon.

13. The silicone oil composition according to claim 12, wherein the crosslinkable composition further comprises (D) an organopolysiloxane that has at least one alkenyl group in each molecule.

14. The silicone oil composition according to claim 12, wherein component (A) is a diene or oligomer thereof or is a polyether.

15. The silicone oil composition according to claim 12, wherein component (B) is an organohydrogenpolysiloxane.

16. The silicone oil composition according to claim 12, wherein the alkyl-modified silicone oil is represented by the following average formula 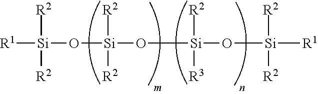
wherein R¹ is an alkyl group having no more than 3 carbons, an alkyl group having at least 4 carbons, an aryl group, or a halogenated alkyl group; R² is an alkyl group having no more than 3 carbons, an aryl group, or a halogenated alkyl group; R³ is an alkyl group having at least 4 carbons; and m and n are each zero or a positive number wherein when n is zero, at least one of R¹ is an alkyl group having at least 4 carbons.

17. A cosmetic ingredient or a cosmetic material comprising the silicone oil composition according to claim 12.
